(19) United States
(12) Patent Application Publication
(10) Pub. No.: US 2014/0261413 A1
(43) Pub. Date: Sep. 18, 2014

(54) GAS DELIVERY SYSTEM FOR RESPIRATORY TREATMENTS OF PATIENTS

(71) Applicant: Tyrene Gibson, Fort Lee, NJ (US)

(72) Inventor: Tyrene Gibson, Fort Lee, NJ (US)

(21) Appl. No.: 14/204,156

(22) Filed: Mar. 11, 2014

Related U.S. Application Data
(60) Provisional application No. 61/851,948, filed on Mar. 14, 2013.

(51) Int. Cl. A61M 15/00

(52) U.S. Cl. CPC A61M 15/0001; A61M 15/009
USPC 128/203.12

(57) ABSTRACT

An improved ear-mounted oral-nasal respiratory device for delivering oxygen and other therapy gases for respiratory treatments of patients. The device comprises an anchoring system for affixing the device to one of the patient's ears and a support arm for placing a gas-conveying nebulizer in the vicinity of the patient's mouth or nostrils.

This application claims the benefit of U.S. Provisional Application No. 61/851,948, filed Mar. 14, 2013.

### FIELD OF THE INVENTION

The present invention is generally in the technical field of medical devices, and, more particularly, is in the field of respiratory devices. The invention is concerned with an improved ear-mounted oral-nasal respiratory device for delivering oxygen and other therapy gases for respiratory treatments of patients.

### BACKGROUND OF THE INVENTION

Oxygen is used to treat respiratory disorders that are wide spread and affect people on all stages of their lives. A common use of oxygen is in treating patients with chronic obstructive pulmonary disease (COPD), asthma, chronic bronchitis or emphysema, people suffering from breathlessness, and trauma patients, among others. Even patients who are not suffering from respiratory disorders directly are often prescribed oxygen in order to compensate for their inability to breathe normally during periods of elevated levels of pain or discomfort.

Many patients require only a supplementary level of oxygen and other therapy gases rather than pure or near pure gases. Such supplementary levels are delivered through a variety of devices depending on the situation, flow of gases required and, in some instances, patients' preferences.

There are two major groups of devices used for delivering supplemental oxygen. One group is represented by so-called nasal cannulas, which typically comprise lightweight tubing routed from a patient's nose, around the patient's both ears and connected to a source of oxygen or other therapy gases. In some cases, such tubing is affixed to the patient's head by a headband. Oral or nasal cannulas are capable of supplying oxygen and other therapy gases at flow rates of 2-6 liters per minute, with a concentration of 24-40%. Nasal cannulas are safe, simple, inexpensive and disposable. In addition, they do not impede patients' ability to eat or drink. Major disadvantages of nasal cannulas, however, are that they cannot be used to treat mouth-breathing patients; they dislodge easily (especially during sleep), and may cause skin irritation, bleeding, fatigue, and tiredness.

The other major group of devices for delivering oxygen is represented by face masks capable of delivering between 5 to 8 liters per minute, with a concentration of oxygen between 28% and 50%. Face masks are usually strapped over the nose and mouth of the patient and provide controllable amounts of oxygen at higher concentrations comparing to nasal cannulas, but they are uncomfortable, carry a risk of suffocation due to rebreathing of CO₂, require constant monitoring, impede eating and drinking, and may cause anxiety and claustrophobic reactions. In addition, both groups of devices are extremely kids-unfriendly: children resist wearing cannulas and face masks because these devices are too restrictive, uncomfortable, and unaesthetic; simply put, they are “not cool”.

Generally speaking, targeted delivery of therapy gases into the nostrils or mouth of the patient increases the effectiveness of the treatment. In some cases, however, untargeted delivery may be suitable for treatments of respiratory distresses of a minimal severity, or for treatments that require low dosages of respiratory medications. For such an untargeted delivery, therapy gases typically flow through a one-way valve to prevent rebreathing, then through cannulas located near or in the patient's nostrils or mouth.

Thus, the choice of a delivery system depends upon the clinical status of the patient and the desired treatment. In addition, children may become frightened or agitated when oxygen is administered, causing their clinical conditions to deteriorate. Therefore, children should remain in a position of comfort whenever possible. It is important to have a system that is up to date with the current needs of the patients.

Recently, there have been several attempts to offer solutions aimed at creating lightweight, safe and convenient supplemental oxygen delivery systems that are suitable for treatment of mouth- and nose-breathing patients. These solutions, however, have not addressed a key requirement, which is a highly adjustable, portable, and child-friendly gas delivery device that does not restrict patient's controllable and uncontrollable movements.

One such solution is the invention described in the U.S. Pat. No. 4,753,233 (the '233 patent), which details a low bulk nasal cannula that utilizes a gas supply line looped around one ear of the patient. Although the '233 patent indeed utilizes only one line and, thus, reduces the patient's discomfort, it, nonetheless, cannot be used to treat mouth-breathing patients. In addition, the flexibility of tubing is further restricted by a wire inserted into the base of the tubing, and because the device is not firmly affixed to the patient's body, it is highly possible for the apparatus described in the '233 patent to be accidentally removed, particularly, during sleep.

Of a background interest is also the U.S. patent application Ser. No. 13/829,044 that describes and illustrates a supplemental oxygen delivery system (the '044 application). Despite its recent filing date, the delivery component of the '044 invention is implemented through either a facemask or a nasal cannula, which inherits all the limitations of its predecessors.

There have been earlier attempts to make traditional nasal cannula devices more children-friendly. For example, the U.S. patent application Ser. No. 13/487,187 (the '187 application) describes a nasal interface adapted for use with generic nasal cannulas. The interface improves oxygen supply by restricting movements of cannulas in a child's nostrils. Thus, unlike the present invention, the invention of the '187 application achieves its goals by being even more restrictive comparing to traditional nasal cannulas, and, therefore, is less comfortable.

The Nasal Ventilation Cannula System and Methods claimed in the U.S. patent application Ser. No. 13/628,038 (the '038 application) offers a positioning system that facilitates placement of a gas delivery nozzle in an optimal location to improve the patient's comfort. The device of the '038 application, however, still relies on the old principle of tubing being rounded over the patient's ears, thus, causing the same level of discomfort as traditional cannulas.

Prior art that most closely resembles the present invention is the Patient Oxygen Delivery System described in the U.S. Pat. No. 6,450,166 (the '166 patent). The '166 patent presents a lightweight oxygen delivery system comprising a curved resilient headband extended from side to side over a patient's head and a clip is secured toward one end of the headband. An elongated tubular boom is secured at one end to the clip to extend and hold its position, when in operation from said one end at the clip to another end located at a space in front of, and proximal to the patient's nose and mouth. In addition, the '166 patent describes an optional embodiment of securing the boom by means of a conventional over-the-ear mount. Although on its face the '166 patent appears to be similar to the present invention, there is an abundance of differences. First, the present invention is a dual device capable of delivering supplemental oxygen as well as providing respiratory treatment therapy. Second, the invention described in the '166 patent utilizes a headband positioned across the top or behind of the patients head, which is not suitable for bed-confined patients. Third, the boom of the '166 invention is not movable, whereas the supporting arm of the present invention is both movable and extendible. Forth, the present invention comprises a unique single point anchoring system that provides superior support to the supporting arm and allows ear-to-nose and ear-to-mouth adjustments based on the patient's preferences and needs; contrary, the '166 patent offers no such functionality. Fifth, the present invention offers a detachable device, which can be manufactured in different sizes and easily sanitized. A unique sleek design is child-friendly and assures that the devise will not interfere with the patient's activities such as walking, sleeping, or being in reclining or horizontal positions. And last, but not least, the present devise comprises a valve/metering component and is equipped with a failure monitor alerting of inadequate gas pressure or dislodgements.

### SUMMARY OF THE INVENTION

It is the general object of the present invention to avoid and overcome the foregoing and other difficulties of and objections to prior art practices by the provision of a child-friendly portable and detachable supplemental gas delivery device that is effective, lightweight, safe, comfortable, and convenient.

Through the personal experiences of her teenage daughter, the inventor has recognized the inherent problems in the art and observed that there is an urgent need for an oral-nasal cannula that is free of disadvantages described above. Thus, the purpose of the present invention is to deliver oxygen and other gases used in respiratory treatments of patients in a more comfortable and less restrictive fashion that has been possible with conventional devices. The present invention is capable of providing adequate gas flow and offers an ability to deliver respiratory treatments to patients in need of such treatments.

The present invention improves upon a typical oral-nasal cannula by integrating an adjustable supporting arm into a single support structure comprising (a) curved ear hook resting on a patient's ear elongated into an adjustable supporting arm with at least one, but preferably several pivot points for allowing adjustments of the supporting arm itself and an oxygen nebulizer attached to said supporting arm; (b) a single oxygen or therapy gas supply line embedded within the supporting arm with one end connected to the nebulizer and the other end connected to the oxygen or gas source through a gas nozzle located at the rear segment of the of the ear hook; (c) the nebulizer flexibly attached to the supporting arm and hermetically connected to the supply line; and (d) a metering component equipped with a failure monitor to alert for inadequate gas pressure or dislodgements.

Thus, the inventor has realized the advantages and benefits of providing a lightweight device worn over one of the patient's ears together with a gas supply line capable of reaching the patient's mouth and nostrils while covering only one side of the patient's face.

### BRIEF DESCRIPTION OF DRAWINGS

These and other features of this invention will be more readily understood from the following detailed description of the various aspects of the invention taken in conjunction with the accompanying drawings in which:

FIG. 1 is a perspective and side view of the therapy gas delivery system;

FIG. 2 depicts the gas nozzle portion;

FIG. 3 depicts the nebulizer portion;

While the invention will be described in conjunction with illustrated embodiments, it will be understood that it is not intended to limit the invention to such embodiments. On the contrary, it is intended to cover all the alternatives, modifications and equivalents as may be included within the spirit and scope of the invention as defined by the appended claims.

### DETAILED DESCRIPTION OF THE INVENTION

The following is a detailed description of the invention provided to aid those skilled in the art in practicing in the field of the present invention. Those of ordinary skill in the art may make modifications and variations in the embodiments described herein without departing from the spirit or scope of the present invention. Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. All publications, patent applications, patents, figures and other references mentioned herein are expressly incorporated by reference in their entirety.

The present invention describes an oral-nasal cannula device (hereinafter “the device”), which holds the nebulizer portion against the patient's mouth or nostrils by means of support affixed to one of the patient's ears.

Referring to FIG. 1, side and perspective views of the device is disclosed. The device comprises a curved ear anchoring hook 102 that positions the device behind the patient's ear 104. Said hook is elongated into an adjustable supporting arm 106 extended to the patient's nostrils or mouth and comprising at least one pivot points 112 for allowing adjustments of said supporting arm itself and a gas nebulizer 114 attached to the supporting arm. Furthermore, a single therapy gas line 116 is embedded within said supporting arm 106 with one end connected to the nebulizer 114 and the other end connected to a source of gas through a gas nozzle located at the rear section of the ear hook 102. Said nebulizer 114 is flexibly attached to the supporting arm 106 and hermetically connected to the therapy gas line 116.

Further referring to FIG. 1, disclosed is an exemplary embodiment that includes at least one but preferably several pivot points 112 along the supporting arm 106. Said pivot points are utilized for positioning and maintaining the nebulizer 114 at the desired location. Other embodiments may forego the dedicated pivots point and accomplish positioning of the nebulizer 114 by other means, including, but limited to, use of a tubing material molded into a desired form, shape and size. In some embodiments, said supporting arm 106 may be implemented as an attachable and disposable component; thus, further improving the cost of the device and its usability.

Referring now to FIG. 2, in some exemplary embodiments, gas nozzle 202 comprises a straight cylindrical tube 204, with a threaded outer tube 206. In another exemplary embodiment said nozzle comprises a small barbed connector. Said gas nozzle 202 is sealed directly into the rear section of said ear anchoring hook and hermetically connected to the embedded therapy gas supply line (not shown). A therapy gas source is removably coupled with said gas nozzle 202. In some exemplary embodiments, an outer end of the rear section of said curved ear hook is formed into a bulbous region 208, such that it offers additional stability when inserted into the patient's ear. It is anticipated that the bulbous region 208 be equipped with a mini-speaker to offer patients additional benefits of audio therapy, such as transmission of music, audio books, etc.

In some exemplary embodiments, the device comprises a metering component. Said metering component utilizes a smart chip and performs system management functions by continually auditing the entire gas delivery device and providing for system integration, ballast, computing, and communication.

In some exemplary embodiments, the device comprises a failure monitor component. Said failure monitor component is based on an in situ gas-delivery monitoring device that proactively measures and records gas delivery rates and pressure. The primary focus of the failure monitor component is twofold as it alerts of two types of events: 1) “no gas”; and 2) “blockage”. In some embodiments, the failure monitor component is integrated into the nebulizer.

In some exemplary embodiments, the device comprises an “inadequate gas delivery” component, which is an in situ monitoring device that monitors gas flow as well as gas quality. A ball float is utilized for displaying gas volume and rate, and a color indicator for displaying gas quality.

In some exemplary embodiments, the device comprises a “dislodged nebulizer” sensor. Said sensor is based on an electronic coupling device capable of determining location of the nebulizer in relation to the patient's nostrils or mouth. In some embodiments, said sensor measures the actual distance. In other embodiments, it relies on secondary indicators, such as relative humidity. Yet in other embodiments a nasal ring may be used to provide conductivity and actual mechanical anchorage for the nebulizer. Alarm sets off when the location of the nebulizer is changed.

Referring now to FIG. 3, in some exemplary embodiments, the nebulizer 302 is substantially spherical in shape and comprises a plurality of radially positioned through orifices 304 for distributing oxygen or other respiratory gases from the therapy gas supply line connected to the nebulizer (not shown) and into the area surrounding the patient's nostrils or mouth, The exact number of orifices 304 in nebulizer 302 depends on the type of therapy and specific purposes of treatment.

Further referring to FIG. 3, in some exemplary embodiments the supporting arm 306 can be adjusted up or down by the patient manually repositioning the supporting arm.

The device is preferably manufactured in a range of sizes to accommodate patients of different ages, genders and body types. The device is manufactured using lightweight plastic or non-corroding metals that are capable of withstanding sanitizing treatments.

The forgoing embodiments have been presented for purposes of illustration and description. They are not intended to be exhaustive or to limit the precise forms disclosed and many modifications and variations are possible in light of the above teachings. The embodiments were chosen and described in order to best explain principles and practical application to enable others skilled in the art to be utilize the various embodiments with various modifications as are suited to the particular use contemplated.

We claim:

1. An ear-mounted oral-nasal respiratory device comprising:
a curved ear hook having a front section and a rear section; said front section is elongated into an adjustable supporting arm, said rear section further comprises an outer end and a gas nozzle for coupling said device to a source of therapy gases;
said adjustable supporting arm further comprising a plurality of pivot points and a front end;
a nebulizer flexibly attached to said front end of said adjustable supporting arm, said nebulizer further comprises a plurality of through orifices.
a therapy gas supply line embedded within said adjustable supporting arm and said curved ear hook having a front end and a back end, said back end is hermetically coupled to said gas nozzle, said front end is hermetically coupled to said nebulizer;

2. The respiratory device as in claim 1, wherein said gas nozzle further comprises a straight cylindrical tube with a threaded outer tube.

3. The respiratory device as in claim 1, wherein said gas nozzle further comprises a barbed connector.

4. The respiratory device as in claim 1, wherein said outer end of said rear section is formed into a bulbous region.

5. The respiratory device as in claim 4, wherein said bulbous region further comprises a mini-speaker.

6. The respiratory device as in claim 1, wherein said nebulizer having substantially rounded outer surface.

7. The respiratory device as in claim 6, wherein said through orifices are radially positioned onto said substantially rounded outer surface.

8. The respiratory device as in claim 1 further comprising a metering component.

9. The respiratory device as in claim 1 further comprising a failure monitor component.

10. The respiratory device as in claim 1 further comprising an inadequate gas delivery component.

11. The respiratory device as in claim 1 further comprising a dislodged nebulizer sensor.

12. An ear-mounted oral-nasal respiratory device comprising:
a curved ear hook having a front section and a rear section; said front section is elongated into a molded tubular supporting arm, said rear section further comprises an outer end and a gas nozzle for coupling said device to a source of therapy gases;
said molded tubular supporting arm further comprises a front end and a nebulizer affixed and hermetically coupled to said front end, said nebulizer further comprises a plurality of through orifices;
a therapy gas supply line having a front end and a back end embedded within said curved ear hook, said front end of said embedded gas supply line is hermetically coupled to said molded tubular supporting arm;
a gas nozzle sealed into said rear section of said curved ear hook and hermetically coupled to said back end of said embedded therapy gas supply line.

13. The respiratory device as in claim 12, wherein said outer end of said rear section is formed into a bulbous region.

14. The respiratory device as in claim 13, wherein said bulbous region further comprises a mini-speaker.

15. The respiratory device as in claim 12, wherein said molded tubular supporting arm is removably coupled to said front section of said curved ear hook.

16. The respiratory device as in claim 12 further comprising a metering component.

17. The respiratory device as in claim 12 further comprising a failure monitor component.

18. The respiratory device as in claim 12 further comprising an inadequate gas delivery component.

19. The respiratory device as in claim 12 further comprising a dislodged nebulizer sensor.
